(19) United States
(12) Patent Application Publication
(10) Pub. No.: US 2011/0245841 A1
(43) Pub. Date: Oct. 6, 2011

(54) EXPANDABLE DEVICES AND METHODS OF USE

(75) Inventors: Shaul Shohat, Kfar Oranim (IL); Idan Tamir, Zichron Ya'akov (IL); Ido Kilemnik, Herzlia (IL)

(73) Assignee: Xenolith Medical Ltd., Kiriat-Gat (IL)

(21) Appl. No.: 13/075,648

(22) Filed: Mar. 30, 2011

Related U.S. Application Data
(60) Provisional application No. 61/319,931, filed on Apr. 1, 2010.

(51) Int. Cl. A61B 17/22

(52) U.S. Cl. 606/127

(57) ABSTRACT

A sieving device and related methods including an expandable sieve mounted on a surgical guide wire. The expandable sieve may be a self-expandable braided filter which is mounted on the guide wire in an axially fixed position, or moveable in one or more directions. The sieving device is deployable in an obstructed ureter, or other body lumen, such as at the beginning of a stone removal procedure, prior to actual stone defragmentation (lithotripsy) phase. The expandable sieve may be set distal to the obstructive stone and expanded to span the entire local ureter cross section in order to retain stone fragments larger than a predetermined size from migrating distally towards the kidney under high irrigation rates/pressures during lithotripsy.

### CROSS-REFERENCES TO RELATED APPLICATIONS

The present application claims the benefit under 35 U.S.C. §119(e) of provisional application Ser. No. 61/319,931 filed Apr. 1, 2010, the contents of which are hereby incorporated herein by reference in their entirety.

### BACKGROUND OF THE INVENTION

Embodiments and methods of the invention may find applicability in the field of ureteral stone retention and retrieval, and other procedures and devices used for preventing or diminishing the migration of harmful debris and other particulates in vessels of the body to organs, such as the kidney, during medical procedures such as, for example, endourological procedures and the like.

Calculi, stones and the like that are formed in body passages, such as in the ureter and the kidneys are a common problem affecting between 5-7% of women and 10-12% men in populations worldwide. These formations are usually caused due to increase in the concentration of urine salts resulting in their crystallization. Kidney stones smaller than 4 mm are usually self-expelled, while larger ones tend to obstruct the ureter causing great pain and interfere with the passage of urine. The disease manifests itself in a wide spectrum of symptoms from asymptomatic to intense pain that occurs as a result of stone fragments making their way down the urinary tract, recurring infections, and damage to the kidney, which can include reduced to complete loss of kidney function.

Until the 1980's, the common treatments for kidney stone removal involved painful and invasive procedures. Since then, kidney stone removal procedures have dramatically evolved to include Extracorporeal Shock Wave Lithotripsy (ESWL), a non-invasive procedure that uses externally-applied shock waves to pulverize kidney stones, and ureteroscopy, a minimally-invasive endoscopic procedure that locates and removes kidney stones through their capture and/or pulverization by holmium laser energy (the latter is termed Intracorporeal Shock Wave Lithotripsy—ISWL).

When the obstructing stone is relatively large, the use of non-invasive techniques to disassemble the stone (e.g. ESWL) is less efficient, so the removal of larger stones typically involves the use of direct concentrated energy to fragment and disintegrate the stones. Such devices may use laser, ultrasonic, electro-shocks, or other fragmenting means. Holmium lasers have been found to be particularly efficient in the field. Typically, the energy source is brought to the target location under endoscopic guidance using a ureteroscope.

A large percentage of ureteroscopic kidney stone removal procedures involve usage of stone baskets for stone capture and retrieval through the ureteroscope's working channel. In the past, stone baskets have been used to extract entire stones, but with the advent of lithotripsy the use of such baskets has been reduced to extracting hard stones that proved refractory to disintegration by lithotripsy, and to collecting large stone fragments. In the latter case, the disadvantage is that the stone basket is introduced after kidney stone disintegration and, therefore, requires either the retrieval of the laser to allow its introduction, or the existence of an additional working channel in the ureteroscope. Clearly, the introduction of stone baskets after stone fragmentation cannot prevent distal migration of fragments towards the kidney during the fragmentation procedure itself. This may be compounded by the use of high irrigation flow typically needed for ureteroscopic imaging.

These circumstances can lead to a cumbersome procedure that frequently fails in retrieving all stone fragments, resulting in translocation of the fragments up the ureter and, at times, into the kidney. This commonly necessitates a longer and more aggressive procedure for the retrieval of such stone fragments by the endourologist, which many times results in increased damage to ureter walls and therefore to longer patient recovery time. This increased damage may also require stenting of the ureter, for example by a temporary double J-stent that prevents ureter wall collapse post ureteroscopy. In such circumstances, an additional ureteroscopic post-operative procedure for the removal of these stents may be required.

Thus, the typical employment of a stone basket post-fragmentation can lead to a prolonged medical procedure; increased intrusiveness; and additional health risks for the patient. These factors result in extended required healing times, and may sometimes necessitate follow-up interventions for removing remaining stones.

Nonetheless, ureteroscopic procedures have gained wide acceptance and become the standard for kidney stone removal. In past years, some attempts have been made to introduce stone fragment capturing devices before and during lithotripsy and then optionally deploying them to remove the fragments from the ureter. These include devices such as the Stone Cone™ Retrieval Coil marketed by Boston Scientific and the NTrap® Stone Entrapment and Extraction Device marketed by Cook Medical. However, none of these devices allow continuous conformation to the narrowing ureter lumen along the extraction path, so fragments may still migrate. Furthermore, these devices may not be applicable for filtering fragments of different sizes, thereby potentially causing less efficient ureteroscopic visualization and irrigation protocols. Additionally, current devices are not applicable for comprehensive filtering but, rather, are used for retaining relatively large stones for direct retrieval. For example, PercSys's Accordion® and Pluromed's BackStop™ are examples of stone retrieval devices. The first is a Nitinol frame-supported fabric that obstructs the ureter, but is provided in discreet sizes (7 and 10 mm) that restrict the ability to fully conform to the ureter's variable diameter. The second is based on Pluromed's proprietary Rapid Transition Polymers™ (RTP™) that are liquid at low temperature and transition to gel at body temperature; the transition is reversible via cooling and the gel is completely dissolvable. The BackStop™ gel forms a plug above the stones in the ureter and prevents stone migration during fragmentation. After the stones are fragmented, the gel is dissolved with saline and exits the body.

### BRIEF SUMMARY OF THE INVENTION

The invention provides systems and methods useful for removing obstructions and other debris from body lumen, such as, for example, a ureter and the like. Embodiments of the invention may include medical devices with an elongated support member, such as a wire, a guide wire, throughwire, shaft, etc., and a sheath surrounding at least part of the support member or the like. In embodiments, the sheath may have an outer diameter of, for example, less than or equal to approximately 1.5 mm, or less than or equal to 1.0 mm and/or may be axially moveable in at least one direction along the support member.

One or more expandable sieves may be attached to the support member and contained at least partially in the sheath. According to aspects of the invention, the expandable sieve may be configured to expand from a collapsed state to an expanded state that substantially conforms to a transverse cross section of a body lumen. In embodiments, the expanded state may be conformable to a lumen having a diameter in the range of, for example, approximately 0.5 mm and 15 mm, or approximately 1.0 mm to 13 mm. In embodiments, the device may include at least two expandable sieves mounted to the support member that are configured to be expanded independently from one another.

In embodiments, the expandable sieve may be configured to substantially prevent the passage of particles with a maximum diameter equal or greater than a predetermined size therethrough while in an expanded state, and to substantially allow the passage of particles with a maximum diameter less than the predetermined size while in the expanded state. For example, the expandable sieve may be configured to substantially prevent passage of particles with a maximum diameter greater than, or equal to 1.0 mm, and to substantially allow the passage of particles with a maximum diameter less than 1.0 mm therethrough. In other embodiments, the expandable sieve may be configured to substantially prevent passage of particles with a maximum diameter greater than, or equal to 2.0 mm therethrough, and to substantially allow passage of particles with a maximum diameter less than 2.0 mm therethrough. In embodiments, the expandable sieve may also be adjustably positionable along a length of the support member.

According to further aspects of the invention, the device may be configured to accommodate a stent, or other medical device, being deployed over a free end of the support member and/or past the expandable sieve. In embodiments, the device may be configured to accommodate, for example, a ureteroscope working channel passing thereupon.

In embodiments, the expandable sieve may be configured to maintain a positioning of the expandable sieve along a length of the body lumen, while subjected to an irrigation flow, by only a radial force exerted by the expandable sieve on a wall of the body lumen while the expandable sieve is in the expanded state. In embodiments, the device may be configured to substantially maintain a position of the expandable sieve against an irrigation flow in a range of for example, approximately 10 ml/min to 1.0 liter/min, or approximately 50 ml/min to 100 ml/min, by only a radial force exerted by the expandable sieve on a wall of the body lumen while the expandable sieve is in the expanded state.

In embodiments, device may be configured to maintain a positioning of the expandable sieve along a length of the body lumen, while subjected to an irrigation flow in a range of, for example, approximately 10 ml/min to 1 liter/min, by a radial force exerted by the expandable sieve on a wall of the body lumen, while the expandable sieve is in the expanded state, and a restraining force applied by the support member.

In embodiments, the sheath may be redeployable over the expandable sieve in the expanded state to return the expandable sieve substantially back to the collapsed state. In embodiments, the expandable sieve may have a compressed diameter of, for example, approximately less than 1.0 mm. The expandable sieve may be configured to expand to an expanded state that is adjustable within a range of, for example, approximately 0.5 mm and 15 mm, or 1.0 mm to 13 mm.

According to further aspects of the invention, the expandable sieve maybe configured to maintain an opening size of less than a predetermined diameter, for example, less than 1.0 mm or less than 2.0 mm, throughout an expanded state range of approximately 0.5 mm and 15 mm, or 1.0 mm to 13 mm.

Embodiments may also include anchors of various configurations located at a distal end of the device.

According to further aspects of the invention, devices as described herein may be included in a medical system configured for fragmenting calcified aggregations, such as kidney stones and the like. Exemplary systems may be configured to interface with at least one of a ureteroscope, a fragmentation energy source, and an irrigation source.

According to further aspects of the invention, methods of removing an obstruction, calcified aggregations, and/or other debris from a body may employ devices as described herein and may include, for example, inserting an elongated support member, or the like, along with an expandable sieve into a body lumen, e.g. hollow or tubular organs such as but not limited to: urethra, urinary bladder, ureter, kidney pelvis, gastrointestinal tract, billiary ducts, gallbladder, pancreatic ducts, blood vessels, upper respiratory tract or bronchi, salivary ducts, lacrimal ducts, articulations, bursa etc. Methods may include positioning the expandable sieve in a collapsed state distally of an obstruction, such as a kidney stone, or the like, and withdrawing a sheath to expand the expandable sieve from the collapsed state to an expanded state.

Embodiments may include fragmenting obstructions, calcified aggregations, and the like, such as by fragmentation means such as laser, electrohydraulic, pneumatic, or ultrasonic lithotripter devices, etc.

Embodiments may include inducing a liquid flow in the body lumen and/or straining particulates with a diameter greater than an opening size of the expandable sieve from the liquid flow in the body lumen using the expandable sieve. Embodiments may include maintaining a positioning of the expandable sieve along a length of the body lumen during the straining and/or induced liquid flow by only a radial force exerted by the expandable sieve on a wall of the body lumen.

Embodiments may also include advancing the sheath to return the expandable sieve from the expanded state substantially back to the collapsed state.

Embodiments may include deploying a stent, or other surgical device, to a body lumen over the support member and/or the expandable sieve.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention claimed. The detailed description and the specific examples, however, indicate only preferred embodiments of the invention. Various changes and modifications within the spirit and scope of the invention will become apparent to those skilled in the art from this detailed description.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced. In the drawings:

FIG. 1 is a schematic illustration of a first embodiment of an exemplary device with an expandable sieve constructed according to the principles of the invention.

FIG. 2 depicts further details of the expandable sieve of FIG. 1 in a compressed state.

FIG. 3 depicts further details of the exemplary expandable sieve of FIG. 2 in an expanded state.

FIG. 4 depicts a schematic of an exemplary anchorless expandable sieve of the invention.

FIGS. 5A through 5C depict various embodiments of wire distal ends that may be employed as anchors for the expandable sieves of the invention.

FIG. 6 depicts another embodiment of an exemplary device that may be used with an expandable sieve of the invention as shown in a ureter.

FIG. 7 is a schematic illustration of an exemplary device of the invention with two expandable sieves.

FIG. 8 is a schematic illustration with further details of an exemplary expandable sieve having a manual positioning and expanding device.

FIG. 9 depicts another embodiment of an exemplary device of the invention shown in a ureter having a blockage.

FIG. 10 depicts an exemplary device and expanded expandable sieve according to the invention as shown in a ureter having a blockage.

FIG. 11 depicts a magnified view of an exemplary device of the invention along with an ureteroscope in proximity to a blockage undergoing fragmentation.

FIG. 12 depicts an enlarged view of an exemplary sieve device of the invention that traps a fragment between the device and a vessel wall.

FIG. 13 depicts use of another exemplary device of the invention in proximity to a blockage undergoing fragmentation.

FIG. 14 depicts use of the other exemplary device of the invention with a sheath removed.

FIGS. 15A and 15B depict magnified views of the use of an exemplary device of the invention used for retrieval of fragments.

FIGS. 16A through 16C depict yet further details of various embodiments of expandable sieves having different shaped expanded states.

FIG. 17 depicts yet another exemplary device of the invention, including an umbrella-shaped expandable sieve.

FIG. 18 is a schematic of another exemplary device of the invention, including a detachable expandable sieve.

FIG. 19 is a schematic of another exemplary device of the invention, having a deployable stent being inserted over the wire.

FIG. 20 is a schematic illustration of restraining forces exerted by expandable sieves of the invention.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a sieve” is a reference to one or more sieves and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily Obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals reference similar parts throughout the several views of the drawings.

Moreover, provided immediately below is a “Definition” section, where certain terms relating to the invention are defined specifically. Particular methods, devices, and materials are described, although any methods and materials similar or equivalent to those described herein can be used in the practice or testing of the invention. All references referred to herein are incorporated by reference herein in their entirety.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

The term “patient” as in treatment of “a patient” refers to a mammalian individual afflicted with or prone to a condition, disease or disorder as specified herein, and includes both humans and animals.

The term “sieve” as used herein refers to devices including webs, meshes, perforations, and other assorted combinations of material and openings, through which a liquid mixture including particles of various sizes may be passed to separate particles of a certain size from the liquid mixture. The term broadly includes various sifters, filters, and devices used for similar purposes, unless expressly distinguished. According to embodiments of the invention, particular sieves may be configured to prevent the flow of relatively large and/or harmful particles, such as preventing large calcified particles and fragments from migrating to the kidneys, without accumulating masses of relatively smaller particles and debris that can lead to total occlusion of the filtered cross section. Particular sieves may be configured to allow a higher irrigation flow than conventional filters, which create a higher resistance to fluids passing therethrough. In embodiments, exemplary sieves may be configured to retain particles of particular sizes, for example, particles with a diameter in orange of approximately 0.5-2.0 mm. According to aspects of the invention, designs may retain certain particles without leading to occlusion of a body lumen in which the sieve is positioned and allowing greater overall irrigation flow to be maintained.

The following preferred embodiments may be described in the context of exemplary ureteroscopic procedures for ease of description and understanding. However, the invention is not limited to the specifically described devices and methods, and may be adapted to various clinical applications without departing from the overall scope of the invention. For example, devices and related methods including concepts described herein may be used for preventing migration and or removal of loose bodies, foreign bodies, or concretions such as but not limited to: calcifications, stones, thrombi, loose tissues, plaque fragments, emboli, bone fragments, in any hollow or tubular organs such as but not limited to: urethra, urinary bladder, ureter, kidney pelvis, gastrointestinal tract, billiary ducts, gallbladder, pancreatic ducts, blood vessels, upper respiratory tract or bronchi, salivary ducts, lacrimal ducts, articulations, bursa etc.

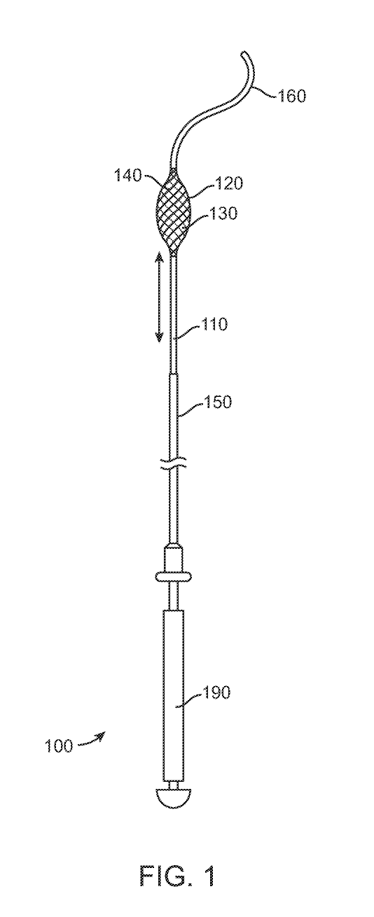

As shown in FIG. 1, a first embodiment of the invention may include a device 100 having a shaft 110, which is connected toward the distal end 160 to an expandable sieve 1120. In embodiments, the shall 110 may be constructed from various materials, and may be configured, at least partially, as a flexible guide wire, and the like, that is conformable to the path of a vessel into which the guide wire is inserted. In embodiments, the expandable sieve 120, and the like, may be fixed to a guide wire, such as shaft 110, or slideably mounted thereto for movement in one or both axial directions along the length of the shaft 110 as shown by arrow in FIG. 1. The expandable sieve 120 is configured to expand to various diameters, e.g. in a range between 0.5 mm and 15 mm, and/or in a range between 1.0 mm and 13 mm, and may substantially conform to a transverse cross section of a body lumen in which the expandable sieve 120 is deployed, as described further herein. Such expandability can be useful in numerous situations. For example, the transverse cross section of the ureter has deviations and changes of diameter along its length, and is also subject to substantial change due to illnesses. Therefore a conforming sieve element such as those described herein may be advantageously used in ureters that range in size, for example, among different patients, along a length of the ureter, and/or according to other physiological circumstances such as illness etc.

As used herein, sieves that substantially conform to a lumen should be understood as those achieving a conformity that further substantially corresponds to an opening size of the sieve. For example, an expandable sieve with an opening size of 1.0 mm that substantially conforms to a lumen will be considered as also having a physical contour, which may be irregularly shaped, presenting no openings between the lumen and the sieve with a minimum diameter greater than 1.0 mm. An opening size of the sieve (130) may be understood as a maximum diameter of openings in the sieve, including, for example, openings in meshed, braided, or porous materials of the sieve, that are configured to allow liquids, and particles smaller than the respective openings, to pass therethrough.

In embodiments, the expandable sieve 120 may be configured as a self-expandable mesh, that is selectively and/or frictionally positionable along a portion of the shaft 110. The portion of the shaft 110 within which the expandable sieve 120 may be positioned, may be bounded, for example by rings, detents, and the like, on the shaft 1110. Such adjustability may be advantageous in selective positioning of the expandable sieve along the length of a vessel. In embodiments, the expandable sieve 120 may be moved in the axial direction by various mechanisms, for example, a positioning wire, and/or a sheath, such as sheath 150 described below.

The device 100 also includes a sheath 150 that is mounted axially about the shaft 110. The sheath 150 may be moveable in the axial direction, and may be configured to at least partially surround the expandable sieve 120. The sheath 150 may include a locking mechanism (not shown) that secures the sheath to the expandable sieve 120, and allows for positioning of the expandable sieve 120 in the axial direction. The sheath 150 may be released from engagement with the expandable sieve 120 to allow the sheath 150 to be withdrawn from the expandable sieve 120.

In embodiments, the proximal end of the shaft 110 may be releasably attached to a handle 190 for manipulating the device 100, adding additional devices over the proximal end of the shaft, etc.

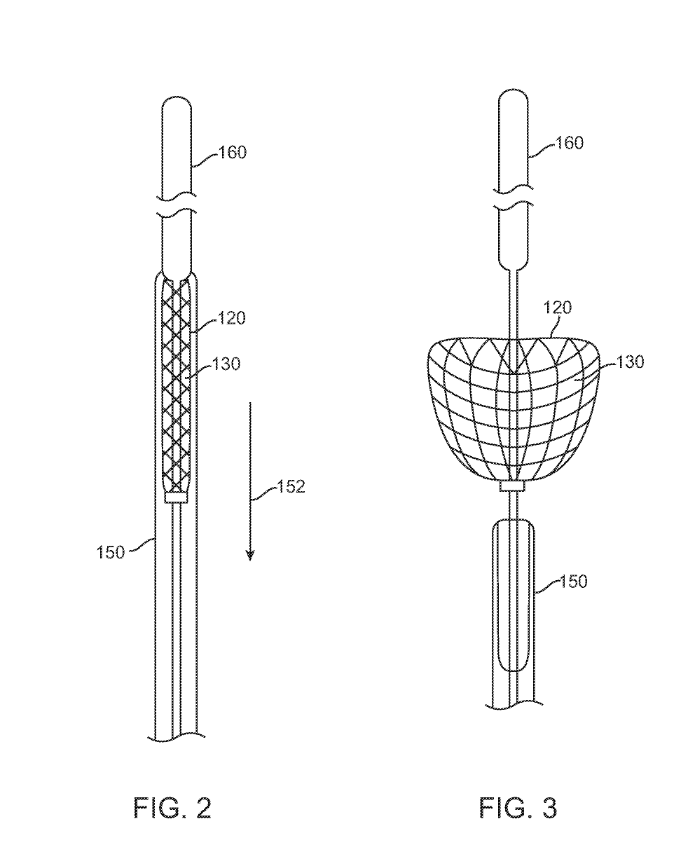

As shown in FIG. 2, the expandable sieve 120 may be contained in sheath 150 such that the sheath 150 maintains the expandable sieve 120 in a compressed state. Expandable sieve 120 may be expanded from a compressed state to an expanded state by various means. For example, expandable sieve 120 may include, or be configured with, a self-expanding mechanism such as a self-expanding compressible weave and the like, or it may be expanded by an active mechanical mechanism, such as, for example, a pull wire or an internal balloon. The expandable sieve 120 may be manufactured of a braided mesh including various materials, and combinations of materials, for example, biocompatible materials such as, but not limited to, Nitinol, Co—Cr alloy, stainless steel and/or other metallic or plastic materials. In embodiments, the expandable sieve 120 may be transitioned from the compressed state, e.g. shown in FIG. 2, to a partially or fully expanded state by, for example, withdrawing the sheath 150 from the covering position around the expandable sieve 120. For example, as shown in FIG. 2, the sheath 150 may be moved in the direction indicated by direction 152 to expose the expandable sieve 120 and remove the restraining force of the sheath 150.

As discussed herein, the expandable sieve 120, and other embodiments of the invention, may include various shapes in compressed and/or expanded states. In some circumstances, an unconstrained shape will be described, which should be understood as the shape that the particular component will assume in the absence of external force acting on the component. For example, the expandable sieve 120 may be configured such that, when released from the sheath 150, the sieve naturally assumes an unconstrained expandable shape that is substantially spherical. Other configurations are also contemplated.

For example, as shown in FIG. 3, the expandable sieve 120 may assume a dome-shaped configuration when the sheath 150 is fully withdrawn from the expandable sieve 120. Such configurations, in which the distal end of the expandable sieve includes the maximum unconstrained diameter, may be beneficial in better allowing the expandable sieve 120 to expand to a greater, and more effective, diameter when the sheath 150 is partially withdrawn. Expandable sieve 120 may be configured to assume various other unconstrained three-dimensional and two-dimensional shapes, including but not limited to, cylindrical ellipsoid, pear shaped, egg shaped, conical, umbrella shaped, tear shaped, and/or circular, elliptical, triangular, etc. In embodiments, the unconstrained shape may have a radial symmetry that may be substantially preserved at various constrained diameters. Such designs may be advantageous, for example, in order to fit various ureteral diameters and geometries, and to substantially obliterate the corresponding lumen with the expandable sieve by closely conforming to the lumen, and may assist in preventing, for example, migration of stone fragments towards the kidney during stone fragmentation.

In embodiments, the expandable sieve 120 may be configured such that the length of the sieve in the collapsed or compressed state is between 50-200 mm, and the length of the sieve in the fully expanded state is between 30-70 mm. In embodiments, the expandable sieve 120 may be configured such that the diameter of the sieve in the collapsed or compressed state is between 0.5-1.0 mm, e.g. 0.9 mm, and the diameter of the sieve in a fully expanded state is between 7-20 mm, e.g. 15 mm. Individual wires of the sieve may be, for example, formed from a metallic, or other material having similar elastic modulus, with a wire diameter of approximately 50-80 μm. In embodiments, an expandable sieve, such as sieve 120, may include approximately 20-72 wires in a braided structure resulting in a suitable range of opening sizes and/or radial pressure when deployed in a body lumen. For example, embodiments may include expandable sieves with configurations that exert a radial pressure of approximately 85-1200 N/m² on a lumen wall when deployed.

As shown in FIG. 3, the expandable sieve 120 may be provided with openings 130, the diameters of which may be configured in a range of for example, 0.5 mm to 2 mm diameter in the expanded state. In embodiments, the openings 130 may be approximately 1 mm in diameter in a partially to fully expanded state of the expandable sieve 120. In embodiments, the expandable sieve may be configured to substantially maintain a maximum diameter of the openings 130 throughout an expanded state range, e.g., a maximum diameter of approximately 1 mm throughout an expanded state range of 1.0 mm to 13 mm, without exceeding a maximal opening size or without deviating from an opening sizes range, such as 0.5 to 2 mm. In some situations, this could include a distribution of opening sizes, all less than the maximal opening size, along a sieve's longitudinal axis at a given state of expansion. However, more uniform opening sizes may be achieved using other configurations, for example, by including multiple layers for the sieve, such as one layer that is woven so its openings change with expansion, and another layer that is porous, non-woven and/or non-elastic that may slide over the first layer without stretching or having fibers movable one with respect to the other. Thus, the openings 130 may be used to substantially prevent stone fragments of a predetermined minimal size and the like to enter into, pass through, and/or to migrate distally to, the expandable sieve 120.

Alternatively, or in combination to the openings 130, the expandable sieve 120 may be provided with a permeable film 140 (see FIG. 1), for example, a liquid permeable film, which covers at least part of the expandable sieve 120, and which prevents stone fragments and the like from entering into, or migrating beyond, the covered portion of expandable sieve 120. An expandable sieve including a permeable film may be used, for example, to reduce a flow of fluid to the kidney pelvis during ureteroscopy and, therefore, reduce the chance for fluid absorption and pain due to distension of the kidney pelvis. Such features may also be used to improve visibility at the stone site. In embodiments, a permeable film, such as film 140, and the like may be configured to be removable from the sieve, whereby the sieving device can be used to present different filtering profiles at different times in the same expanded state. For example, a permeable film may have an opening size of 0.1 mm, which may be advantageous for limiting the flow of smaller debris etc. at certain times such as the initial deployment of the device. The sieve may also have an expandable mesh or the like with a different maximum opening size, e.g. approximately 1 mm. Therefore, after the permeable film is removed, the sieving device may present an opening size that is, for example, an order of magnitude different than the opening size of the device when the permeable film is attached. Various ways of removing a permeable film and the like are contemplated, such as, connecting the film to a separate control wire that can be independently withdrawn along with the film.

The sheath 150 may be configured as a hollow tube with dimensions including, for example, an outer diameter equal to or less than 1.5 mm, or, optionally, approximately 1.0 mm. In embodiments, the inner diameter of the sheath 150 may be in a range of 0.3 mm to 1.0 mm, e.g. approximately 0.9 mm, and may be sized according to the shaft 110 and/or a compressed diameter of the expandable sieve 120.

In embodiments, the sheath 150, or similar overtubes and the like, may be removable from the device 100, such as by fully withdrawing the sheath 150 over the proximal end of the device. In further embodiments, the sheath 150 may also be reinstalled over the shaft 110, and/or the expandable sieve 120. As described further herein, such features may be advantageous, for example, in allowing the device 100 to assume a smaller profile within a ureteroscope and the like for a period of time during a procedure, and also allow for the re-compression of the expandable sieve after other tasks, such as, for example, fragmentation and irrigation, are completed, and/or to reposition the expandable sieve.

In other embodiments, a sheath, or similar overtube, may be actuated by a separate pull chord, rather than being directly pulled upon. For example, a partial sheath, that extends only partly along a length of the device, may be remotely actuated, and, in embodiments with multiple expandable sieves, one or more sieves may have separate partial sheaths that can be individually actuated to allow for selective expansion of a particular expandable sieve.

The distal end 160 of the device 100 may take various forms, including, for example, a continuation of a guide wire or the like, extending through the expandable sieve 120, and/or a distal end of the expandable sieve 120 may be attached to a guide wire. In embodiments, guide wires, such as the distal end of the device 100, may include portions that are bendable and/or twistable to form an expanded anchoring portion in a kidney opening.

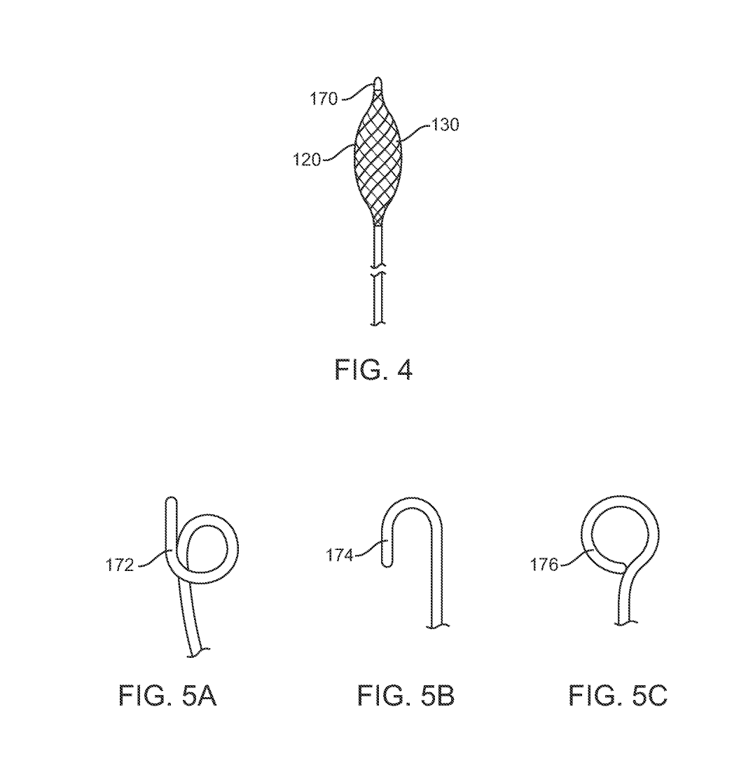

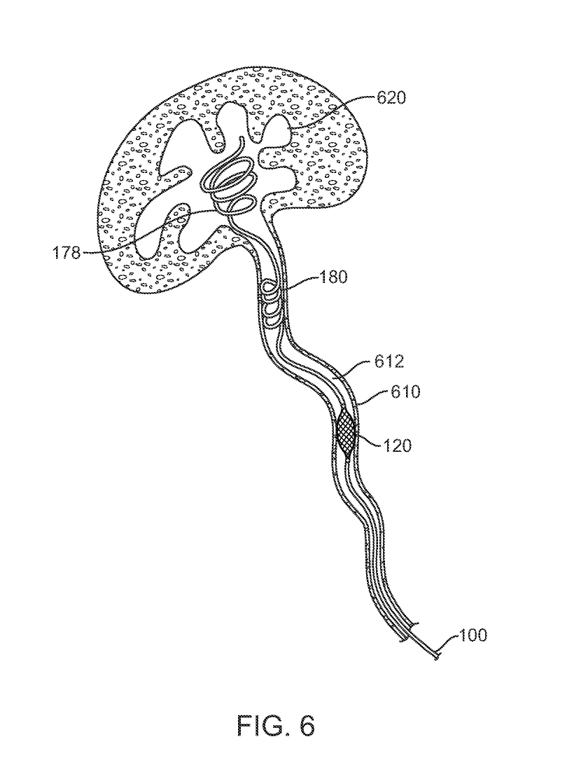

As shown in FIG. 4, the distal end of the expandable sieve 120 may terminate in a anchorless nub 170, or may be attached with various extensions, anchors and/or ends. For example, as shown in FIGS. 5A-5C, the device 100 may terminate with a tip, which may be tapered, flexible, floppy, semi-rigid, hydrophilic, etc. The tip may be pre-configured, and/or dynamically shaped, at its distal end to the form of a pig tail 172, a U-shape 174, or an O-shape 176. Such distal ends of the device 100 and guide wire may serve as a retention means, or anchor, of the guide wire in, for example, the kidney pelvis, and prevent its dislodgement during procedures, such as stone fragment evacuation from the ureter, such as shown in FIG. 6.

FIG. 6 depicts aspects of an exemplary device 100 as may be used in a procedure requiring insertion into a ureter 610, having lumen 612. As shown in FIG. 6, the device 100 is configured to traverse the length of ureter 610 and to enter the kidney pelvis 620. The expandable sieve 120 is located at a position along the length of the ureter 610 within the lumen 612. The device 100 is shown with a distal end including helical shape 178 that may be inserted in the kidney pelvis 610. Additionally, a portion 180 of the proximal end of the guide wire may be configured, such as in a helical pattern, to further prevent dislodgement of the distal end of the guide wire from the kidney pelvis during manipulation of the device. For example, by providing secondary restraining means, such as the helical portion 180, in the ureter, a reduction in force applied to the restraining means in the kidney pelvis, e.g., helical end 178, may be achieved. According to embodiments, sieving devices including expandable meshes and/or braids may also be used to anchor the device in place with, or without, an additional anchor such as those shown in FIGS. 5A-5C and 6.

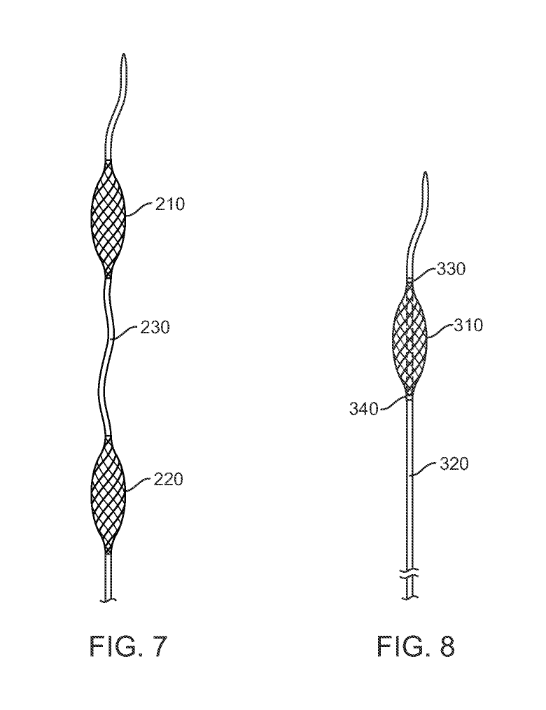

In embodiments, a device may be provided with more than one, e.g. two, expandable sieves. For example, as shown in FIG. 7, a device may include expandable sieves 210 and 220, which may have similar characteristics to expandable sieve 120 depicted in FIGS. 1-3. Expandable sieves 210 and 220 may be connected, for example, by a connecting filament 230 that may be, for example, straight, helical, or other shapes described herein. In embodiments, expandable sieves 210 and 220 may be separately expandable such that each of the expandable sieves 210 and 220 may be expanded at different times and/or amounts. For example, a sheath, such as sheath 150 shown in FIG. 1, may be configured to withdraw from one of the expandable sieves 210 and 220 first, and withdraw from the other of expandable sieves 210 and 220 afterwards. Such configurations may allow, for example, a clinician to position and secure the first expandable sieve at a desired location, and then position and secure the second expandable sieve, at a second desired location, which may be adjustable and/or different than the length of connecting filament 230. More than two expandable sieves are also contemplated and may be achieved using similar methods. Also, one or more expandable sieves may be fixed, and one or more expandable sieves may be adjustable in an axial direction.

Embodiments may also include locking mechanisms whereby the expandable sieve, such as expandable sieve 120 depicted in FIGS. 1-3, may be temporarily or permanently locked in an at least partially expanded state through manual means. For example, as shown in FIG. 8, the expandable sieve 310 may be provided with a locking means, which may include an internal filament 320 connected to a distal end 330 of the expandable sieve 310. The internal filament 320 may be connected to the expandable sieve 310 from within and pass through a proximal orifice 340, or neck, of the expandable sieve 310. In this configuration, the proximal end 340 of the expandable sieve 310 is fixed to the shaft and the distal end 330 generally moves in an axial direction as a diameter of the expandable sieve 310 is expanded or contracted. For example, as the diameter of the expandable sieve 310 is compressed, the length of expandable sieve 310 increases. Therefore, by fixing, or applying sufficient tension to, internal filament 320, the expandable sieve 310 is restrained, or locked, from returning to the compressed, or more slender, configuration. In other embodiments, the internal filament 320, or the like, may be used as a positioning rod to move an expandable sieve (which is mounted on the guide wire and moveable along the axial direction) in one or both axial directions.

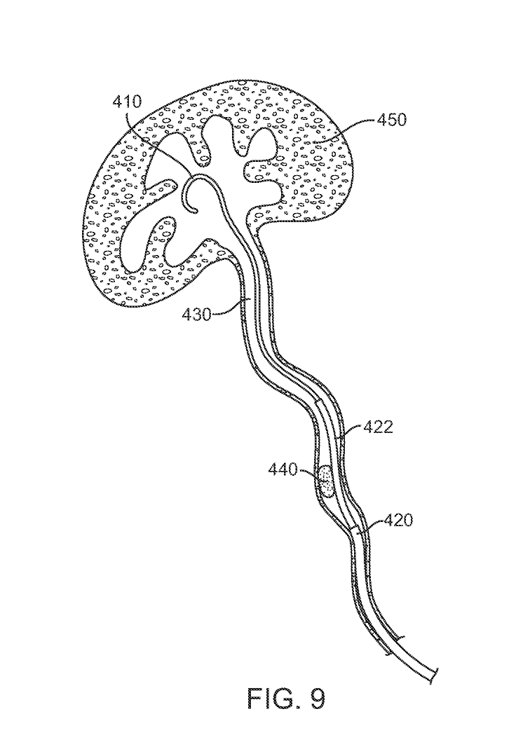

In embodiments, a device of the invention, such as the device 100 depicted in FIG. 1, may be configured for use in a working channel of a ureterscope. For example, components of the device 100, e.g. sheath 150, shaft 110 and any components distal thereto, may be sized and configured to be inserted into a working channel of a ureterscope, and optionally controlled via controls of the ureterscope. In embodiments, and as discussed further herein, various other components may be used in combination with the disclosed devices and ureterscope, such as stents that may be positioned over the device before or after its insertion into a body lumen. Accordingly, stents, and other devices may be deployed, for example, after the expandable sieve is returned to a compressed state, without removing the device from the body lumen. Further details regarding exemplary uses of the disclosed devices and methods are shown with reference to FIG. 9.

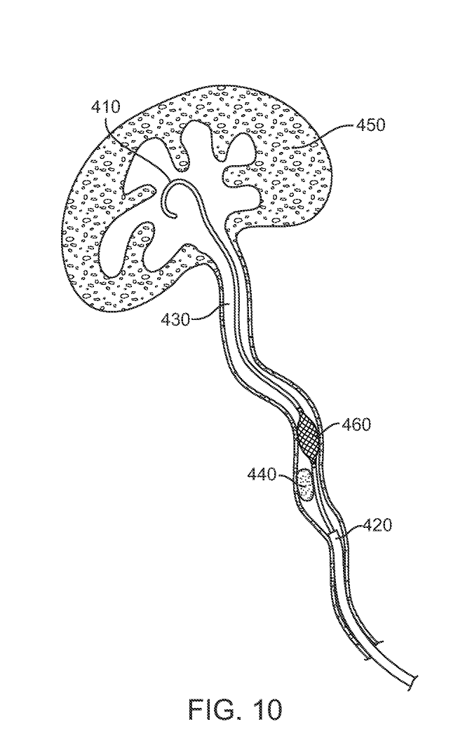

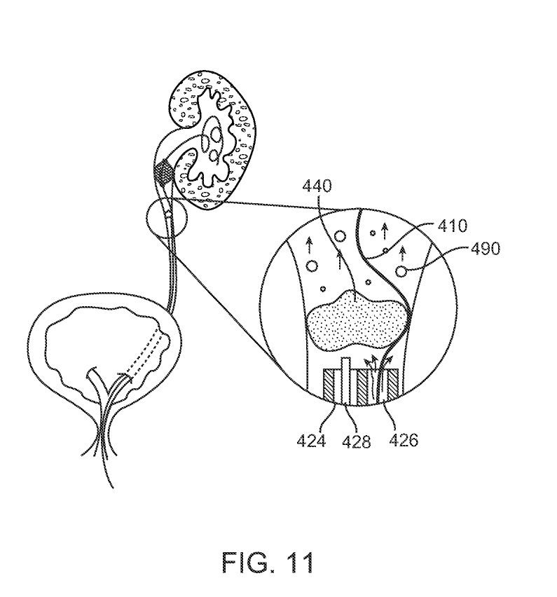

As shown in FIG. 9, a device 410 may be introduced through a ureteroscope 420 working channel, within the ureter 430. In the example shown in FIG. 9, the device 410 is fed past an obstruction 440 in the ureter 430, such as a kidney stone, towards the kidney 450, which may be done, for example, under direct vision and/or fluoroscopy. An expandable sieve (not shown) may be held within the sheath 422 in a compressed state, whereby the expandable sieve may be positioned distal to the obstruction 440. Thus, the sieving device is deployable in an obstructed ureter at the beginning of a stone removal procedure prior to actual stone defragmentation (lithotripsy) phase. As discussed previously, a distal end of the device 410 may be positioned in a kidney pelvis and include means for anchoring the device 410 in the kidney, such as curved, helical and other shaped bends and/or ends. Further details are shown in FIG. 10-11.

After the device, and expandable sieve(s), are positioned properly, the outer sheath 422 may be retracted revealing a self expandable sieve 460 that, as shown in FIG. 10, expands and occupies the ureter 430 above the obstruction 440. The self expandable sieve 460 may expand, for example, up to 30 times its collapsed deployable form (e.g., from about 0.5 mm to about 15 mm in diameter) while exerting mild pressure/force onto the vessel, e.g. ureter, walls in various diameters. One advantage of certain embodiments using a fully retractable and removable outer sheath is that, after the sheath is withdrawn to allow the expandable sieve to manually or self-expand, the now thinner proximal part of the shaft by itself allows more room for irrigation, or other devices, to be run through the ureterscope working channel. Once the filter has been deployed, the clinician can apply irrigation at a higher flow rate, e.g. for improved endoscopic visibility, and the like, thereby allowing better control over the laser. Such irrigation may be applied through the ureteroscope's working channel without introduction of other instruments through the ureter.

As shown in the expanded view in FIG. 11, a portion of the guide wire proximal of the expandable sieve extends past the obstruction 440. An end of the ureteroscope 420 includes two working channels 424 and 426, although other ureteroscopes may be used having one or more than two working channels. The device 410 is routed through working channel 426. In other embodiments, the device may be inserted side-by-side with a ureteroscope.

A fragmentation energy source may be deployed through the same, or different, working channel of the ureterscope. Other actuating means, in addition to the sheath and self-expanding sieve, are also contemplated to expand various different expandable sieves described herein. In the position depicted in FIG. 11, the obstruction 440, such as a kidney stone, may be fragmented by standard fragmentation means such as laser, electro-hydraulic, pneumatic or ultrasonic lithotripter devices used in combination with, or separately from, ureteroscope 420.

For example, as also shown in FIG. 11, a laser fiber 428 may be included in working channel 424 and energized to promote fragmentation of the obstruction 440. Liquid may also be released into the ureter 430 via working channel 426 to promote washing away of the resulting fragments 490. Consequently, fragments 490 may be dislodged from the obstruction 440 and travel up the ureter toward the expanded expandable sieve 460. Thus, as described above, and as will be apparent to those understanding the concepts described throughout the specification, embodiments may include replacing all, or a portion, of a working channel previously occupied by a sleeve, sheath and the like, with irrigation flow, suction flow, and/or additional tools or instruments, such as a fragmentation device and the like. As mentioned previously, devices according to the invention may also be used, instead, side-by-side with a ureteroscope or other device deployed in the body lumen.

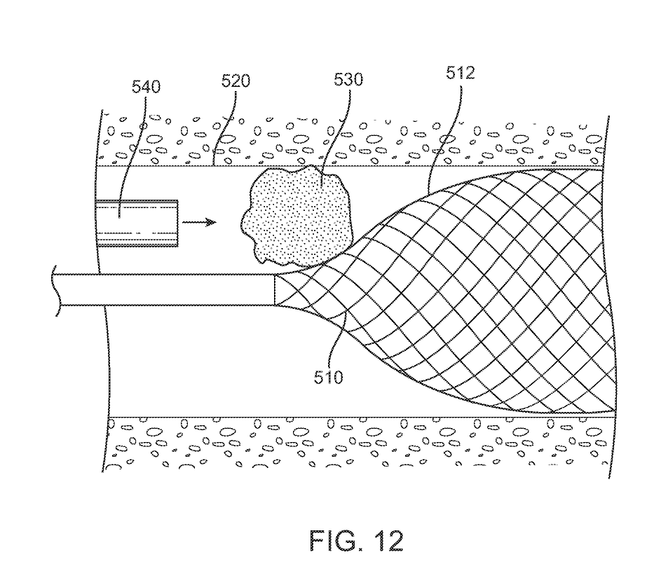

In alternative embodiments, an expandable sieve may be positioned to contact an obstruction (e.g. stone, stone fragment, clot, other solid or semi-solid particle), and to entrap the obstruction between a surface of the expandable sieve and a surface of the body lumen, for example, for the dual purposes of particle retention and subsequent fragmentation. As shown in FIG. 12, an expandable sieve, or trapping mechanism 510, may be positioned near obstruction 530. According to embodiments, the obstruction 530, and the like, may be trapped between an artificial surface and a biological surface, for example, surface 512 of the expandable trapping mechanism 510 and a surface 520 of the body lumen in which the trapping mechanism 510 is deployed. Accordingly, a fragmenting mechanism 540, such as a laser, electro-hydraulic, pneumatic or ultrasonic lithotripter device, etc., may be positioned and applied to the obstruction 530, as well as other fragments that may develop and be subsequently trapped by the expandable trapping mechanism 510.

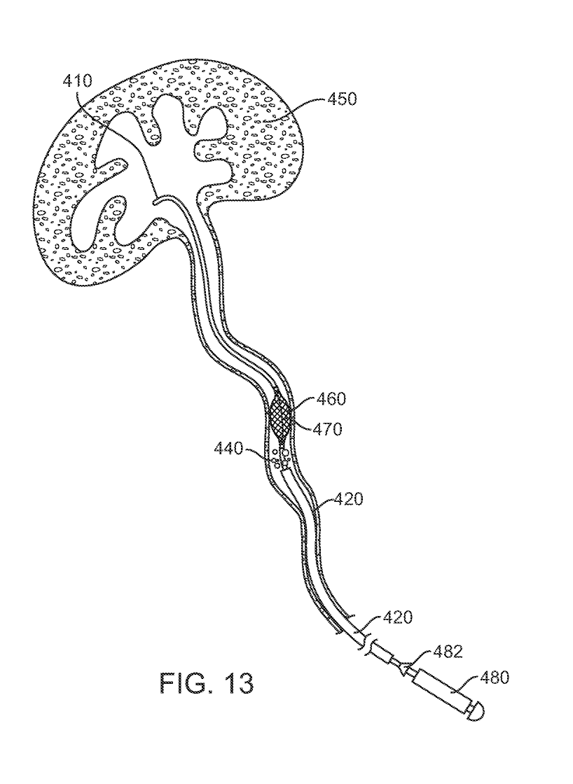

Other possible surfaces upon which obstructions and other particles may be trapped include, for example, biological surfaces including blood, ureter or other vessel walls, and artificial surfaces including braids, meshes, etc. of the expandable sieves and trapping mechanisms described herein. In embodiments, the artificial surface may be a self expandable braid that exerts force against the biological surface, ensuring their apposition, which facilitates particle entrapment. The artificial surface may include a sieve having openings of a size of a specific pre-determined sizes range, allowing corresponding particle fragment migration through it. For example, according to embodiments, the expandable trapping mechanism 510 may include a surface configured to substantially prevent passage of, and to apply a trapping pressure to, particles with a diameter greater than approximately 1.0 mm, or other therapeutically desirable size depending on the nature of the procedure, particle composition, affected vessel, and/or other relevant organs. Further details regarding exemplary sieving and trapping techniques are described with reference to FIG. 13.

As shown in FIG. 13, stone fragments and the like of sizes greater than a predetermined value may be prevented from migrating towards the kidney 450 by the expanded expandable sieve 460 that is situated above the remains of fragments 440. Optionally, the sieve once expanded is configured to allow passage of fragments and particles of, for example, less than 2 mm, or optionally less than 1 mm, therethrough. Optionally, such passage is only applicable under irrigation of minimal flow rate value. Expandable sieve 460 is shown within the ureter and occupying its lumen. It should be understood that the expandable sieve 460 closely conforms to a transverse cross section of the lumen such that fragments are prevented from migrating beyond the expandable sieve 460 without passing through it. For example, the remains of obstruction 440, previously shown in FIG. 11, must be reduced to an appropriate size, e.g. approximately less than 1 mm, in order to pass through the openings of expandable sieve 460. In embodiments, the expandable sieve 460, and the like, may be configured such that no gaps with a minimum diameter greater than 1 mm are present between an outer contour of the expandable sieve and the transverse cross section of the body lumen.

In embodiments, devices according to aspects of the invention may be configured and/or used to withdraw blockages, fragments, debris and the like, in addition to sieving. For example, a device, such as that shown in FIG. 13, may be pulled by the shaft 410, filament 470, or other attached mechanism, and the expanded expandable sieve 460 will brush out fragments remaining in the ureter, or trapped in the expandable sieve. In embodiments, after fragmenting the stone to a desired degree, the expandable sieve, optionally together with the ureteroscope, may be withdrawn. The sieve may then serve as a radially changeable wiper that wipes the large fragments towards direction of pull, e.g. toward the bladder, while adjusting and conforming to changes in the dimensions of the vessel, e.g. the narrowing ureter, as it travels distally. Thus, the device may be withdrawn with the fragments or other material, through the bladder, and ureteroscope or introducer sheath, to the bladder. In embodiments in which the device is provided with a locking device including, for example, filament 470, the device may be pulled after tensioning and/or locking the locking device, which may be used to prevent the expandable means from resuming its more slender configuration. This may allow or a more forceful brushing out of the remaining stone fragments.

The handle 480 and the sheath 482 of the device, shown in FIG. 13, may be removed such that only the shaft 410, or other guide wire, that is connected to the expandable sieve 460, remains within the working channel of the ureteroscope 420, or within the introducer sheath.

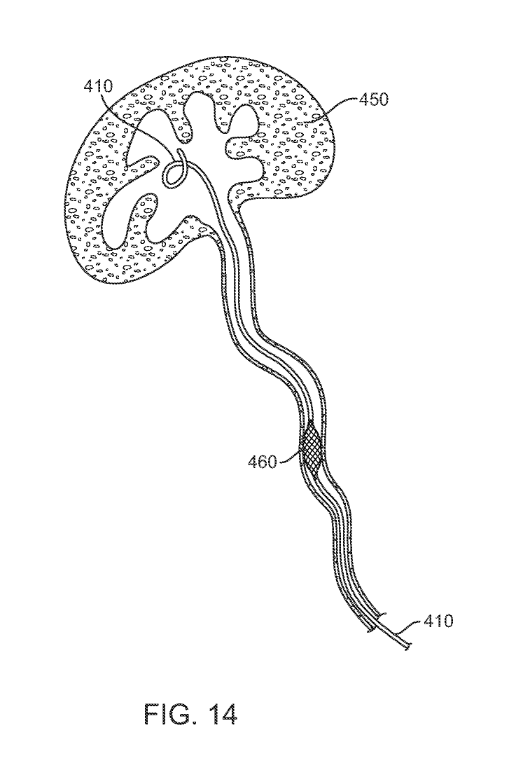

Thus, as shown in FIG. 14, the device may be easily reduced to the shaft 410 and expandable sieve 460. The shaft 410 of the device may have a very small diameter, for example, between 0.5 mm and 0.01 mm, and preferentially, between 0.3 mm to 0.1 mm. Such dimensions may be advantageous in leaving additional space within the working channel, for other instruments, irrigation, etc., and/or for deploying other medical devices, such as stents, over the device.

As previously discussed with respect to FIG. 7, a device including more than one expandable sieves, e.g. expandable sieves 210 and 220, may also be used. Such devices may be introduced into the ureter, extending above the obstruction 440, as previously described. Both of expanding sieves 210 and 220 may be expanded to an at least partially expanded state, whereby the ureter is occupied at a distal location, via expandable sieve 220, and a proximal location, via expandable sieve 210. The distal guide wire 160 my be introduced within the kidney pelvis of kidney 450, and serve as retention means for the device. As discussed previously, an obstruction 440 may be fragmented by various means, or the obstruction 440 may be trapped, captured, and/or withdrawn without fragmentation or other reduction techniques. For example, according to embodiments, the obstruction may be fragmented, and the device may thereafter be pulled, such that the proximal expandable sieve 210 brushes out the ureter including substantially all fragments with a diameter larger than an opening size of the proximal expandable sieve 210. The distal expandable sieve 220 may remain within the ureter while the proximal expandable sieve 210 is withdrawn. Thus, the distal expandable sieve 220 may advantageously prevent possible migration towards the kidney 450 of fragments or other debris that may escape the sweep by proximal expandable sieve 210, such as fragments that lodge in the vessel wall or are otherwise not collected by the expandable sieve due to irregularities and/or contortions in the vessel, etc. In embodiments, an opening size of the distal expandable sieve 220 may be different than an opening size of the proximal expandable sieve 210. For example, the distal expandable sieve 220 may have a smaller opening size than that of the proximal expandable sieve 210, such as to allow for a relatively easier retraction of the proximal expandable sieve 210, while still maintaining a desired filtering size at the distal expandable sieve 220.

In embodiments, a sheath (not shown) may be individually advanced over the proximal expandable sieve 210, and the device pushed again within the ureter, for example, while the distal expandable sieve 220 is still in an expanded state. The proximal expandable sieve 210 may then be re-exposed and expanded by withdrawing the sheath, and additional fragments may be brushed out and/or trapped for further fragmentation or other reduction. This procedure may be repeated as needed and may be particularly suitable in cases involving a large stone burden, or including multiple stones at different locations within the ureter.

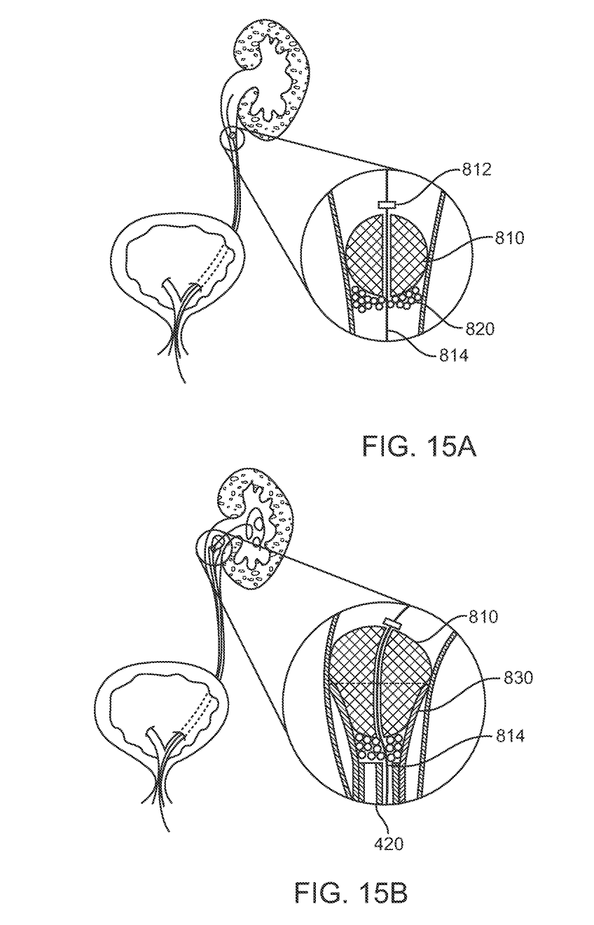

According to yet other aspects of the invention, embodiments may include features and methods that allow for trapping particles while the device is inserted in the body lumen. For example, as shown in FIGS. 15A-15B, an expandable filter 810 may include an optional stopping mechanism 812 that may be used to hold the expandable fitter in a position along length of guide wire 814 or to withdraw the expandable filter 810. It should be noted that, according to embodiments discussed further herein, a stopper 812, or the like, may not be required to maintain a positioning of the sieving device, such as expandable filter 810, which may be configured to maintain its position using radial forces alone.

As shown in FIG. 15A, a collection of fragments 820 have been trapped by expandable filter 810, and are being prevented from migrating toward the kidney. Turning to FIG. 15B, a sleeve 830, such as an access sheath, may be introduced over the guide wire and advanced in the lumen, and/or the expandable filter 810 withdrawn into the sleeve 830, such that the fragments 820 are substantially contained between the expandable filter 810 and the sleeve/access sheath 830. In embodiments, the opening of the sleeve 830 may be expandable and/or adjustable to closely approximate a transverse cross section of the lumen. Once the fragments 820 are substantially contained, the device can be withdrawn to remove the fragments, or the area between the expandable filter 810 and the sleeve 830 can be flushed with irrigation and suction, via the ureteroscope 420, or the like, to remove the fragments. Although various exemplary embodiments are shown with a configuration including a sieve, such as a braid, being passed over a wire, other configurations are also contemplated, for example those in which the sieve attaches at one or more ends with a wire that does not pass through the sieve.

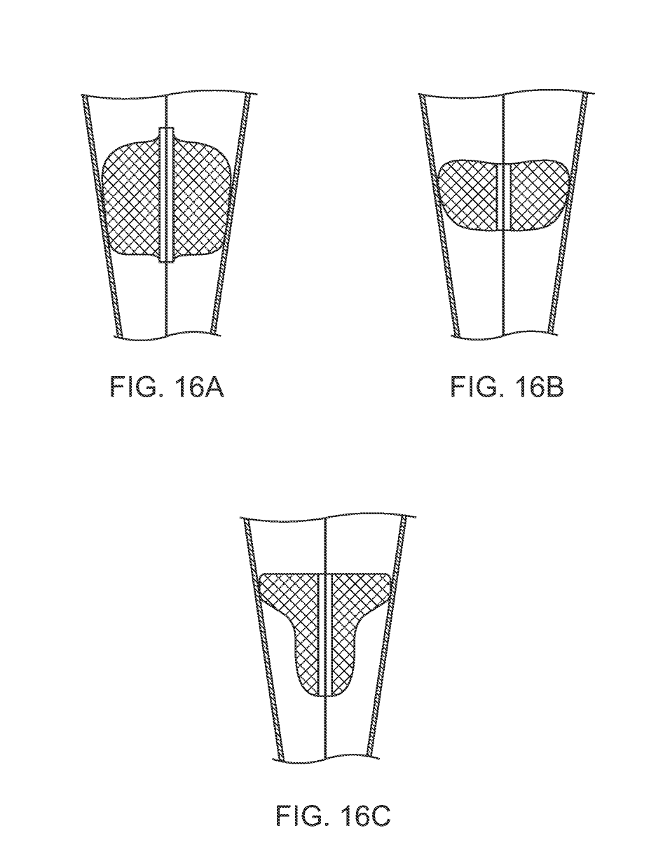

Expandable sieves in accordance with principles described herein may take various forms. For example, as shown in FIG. 16A, the expandable sieves may have a substantially spherical configuration in an unconstrained expanded state, that adopts a cylindrical component along sidewalls when constrained in a body lumen. Other configurations may include a dome-shaped configuration in an unconstrained expanded state, which also adopts a cylindrical component along sidewalls when constrained in a body lumen, as shown in FIG. 16B. Embodiments may include a closed and convex shape at the proximal end and/or an opened, and optionally concave, shape at the distal end. As shown in FIG. 16C, embodiments may include bell-shaped, and other configurations, in which a sieve portion has a contoured shape at its proximal end, which may be used to promote fragment accumulation in specific region(s), e.g. at a sieve periphery, while allowing uninterrupted continuous irrigation flow through its center.

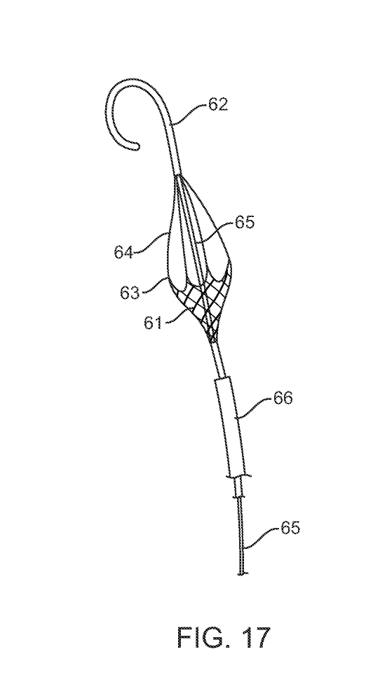

In embodiments depicted in FIG. 17, the expandable filtering means 61 may be conically shaped. Configurations such as shown in FIG. 17 may provide advantages, for example, when varying opening sizes are desired, such as larger openings in the distal end of the sieve. Such configurations may, for example, prevent debris accumulation within portions of the braid that could interfere with stenting over the device, in the embodiment depicted in FIG. 17, the tip 62 faces distally, e.g. towards the kidney, and the distal rim 63 of the expandable filtering means 61 is connected by one or more, and preferably 3, filaments 64. The shaft 65 may be attached from within to the tip of the expandable means. Such a configuration may be employed to act like an umbrella that can be expanded in a way that only requires a liquid flow to pass through one surface of the sieve, rather than two surfaces such as in a spheroid, or other substantially enclosed-shaped, sieve

For example, in embodiments, to expand the expandable filtering means 61, the external sheath 66 may be retracted. The shaft 65 and/or filaments 64 may then be adjusted to control an expansion of the filtering means 61. In embodiments the external sheath 66 may be removed during fragmentation of the stone. When the device is expanded within the ureter above the stone or stone fragments, it can capture them and permit their evacuation.

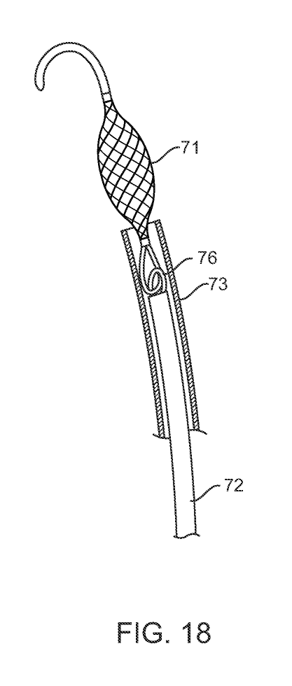

In another embodiment shown in FIG. 18, an expandable device 71 may be detachable from shaft 72, via a securing mechanism 76. Securing mechanism 76 may be, for example, remotely actuated to detach the expandable device 71 from the shaft 72. In embodiments, the expandable device 71 may be configured to be pushed out of an external sheath 73 by shaft 72, in order to, for example, allow the expandable device 71 to undergo a self-expansion to an expanded state. The expanded expandable device 71 may be retrieved by a standard grasper, and the like. Such embodiment may be desirable in order to, for example, prevent stone migration to cavities such as calyxes of the kidney during ureteroscopy and/or percutaneous nephrolithotripsy.

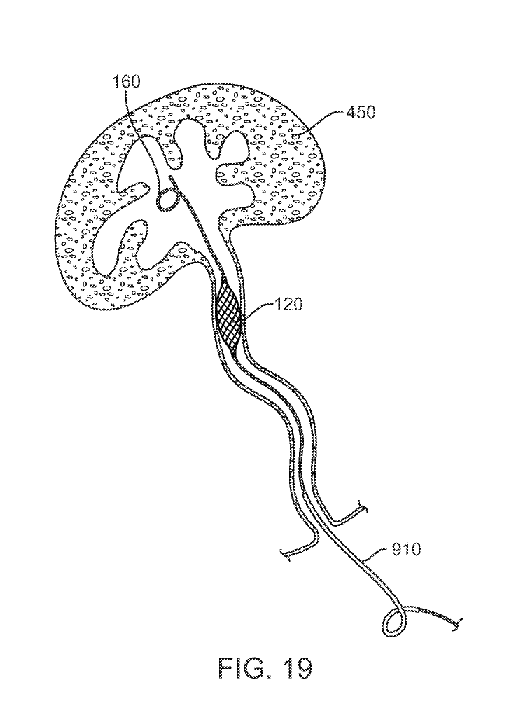

In embodiments, exemplary devices may also serve as a guide wire for introducing and/or guiding intraluminal devices, such as a catheter, an imaging apparatus such as an ureteroscope, and/or a stent, to a position along the device, e.g. over the device to the kidney pelvis (using introduction techniques sometimes referred to as “over-the-wire” and “rapid exchange”). As known in the art, a ureteral stent, or ureteric stent, may be configured as a thin tube that is inserted into the ureter to prevent or treat obstruction of the urine flow from the kidney. The length of the stents used in adult patients my vary between 24 and 30 cm. Such stents, and the like, may be used according to aspects of the invention, for example, as shown in FIG. 19. Embodiments may include configurations in which the device may be readied to accommodate a stent, or the like, such as by removing handles and/or other large-diameter portions of the device, leaving a free end of the device to receive a stent, such as D-J stent 910, over the sheath and/or shaft of the device. Stent 910 may be a hollow tube with a diameter and gauge sized according to the patient's ureter and/or condition.

After being placed over the free end of the device, the stent 910 may be guided to a desired position, such as the renal pelvis. In embodiments, the stent may be advanced by the use of, for example, a push tube (not shown), such as a plastic tube that is advanced along the wire and pushes the stent into place. In certain embodiments, a sheath of the device may be removed and the stent advanced over the shaft of the device, including over the expandable sieve 120, and over the distal guide wire 1160. In alternative embodiments, the sheath, such as sheath 150 shown in FIG. 3 may be advanced over the expandable sieve 120 to re-collapse the expandable sieve 120 before advancing the stent 910 over the sheath 150 and expandable sieve 120.

Thus, according to aspects of the invention, a device may be used, first, to prevent migration of potentially harmful fragments and the like, and, second, to act as a guide for placement of a stent, such as a ureteral stent. By providing a device that can accomplish both of these functions, trauma to the patient may be reduced by minimizing the number of separate devices that need to be inserted and withdrawn from the patient, while also reducing the overall time associated with these procedures as well as the number of ureteroscope entries into the ureter. This type of dual use may be particularly beneficial in procedures such as kidney stone removal and ureteral stent insertion, which may be required together. For example, ureteral stents may be used to ensure the patency of a ureter that has been compromised by a kidney stone. In other situations, it may become necessary to place a stent in the ureter if the ureter has been irritated or scratched during stone fragmentation and/or removal.

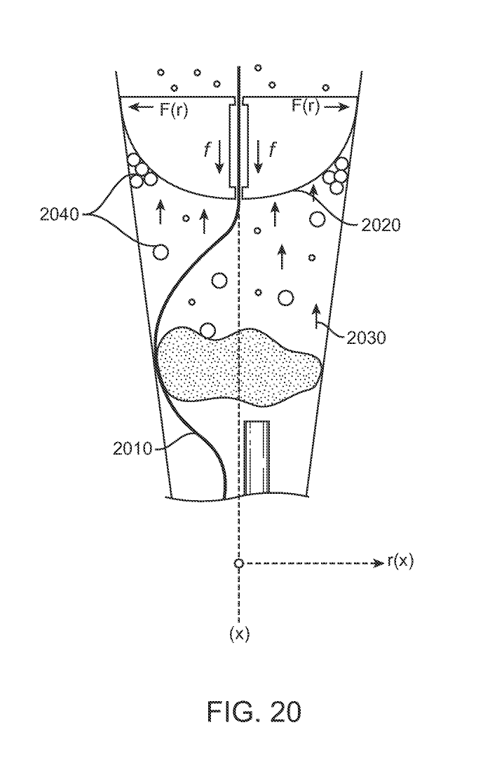

As depicted schematically in FIG. 20, in embodiments a static friction force “f” may be applied to a through wire 2010, such as the guide wire, passing through the expandable sieve 2010 to provide a resistive force and maintain the position of the expandable sieve in the lumen against a flow 2030 containing debris 2040. The expandable sieve 2020 may also be held in place by applying a combined force F+f, wherein “F” is the radial force F(r) applied by the expandable sieve and “f” is the static force f applied by the throughwire 2010. In other embodiments, the sieving device may be configured to substantially maintain its position, and to resist a combined pressure from the flow and debris, through the force F(r) only. That is, the device may be configured to substantially maintain a position during operation without a restraining force/being applied by a throughwire or the like.

According to embodiments, the expandable sieve 2020 may be held in place, by F(r) only, F+f, or f only, while countering a debris containing irrigation flow 2030 at various therapeutic levels. For example, according to embodiments, a force F(r) may be exerted by an expandable sieve to resist flows of approximately 1 liter/min, 100 ml/min, or 10 mil/min. Preferable ranges have been found to be, for example, between 50-100 ml/min. For example, in one embodiment the expandable sieve may be held in place in a flow of 1 liter/min by homogenously exerting outward radial force F(n) towards walls of the body lumen, without a force f applied by the throughwire 2010 or the like. In embodiments, the sieve may be configured to exert a radial force of for example, approximately 85-1200 N/m² when deployed.

The description given above is merely illustrative and is not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments.

1-18. (canceled)

19. A medical device comprising:
an elongated support member;
a sheath configured to surround at least part of the support member, the sheath being axially moveable in at least one direction along the support member; and
a first expandable sieve configured to be deployed in a body lumen and to be held at least temporarily in a compressed state by the sheath,
wherein the first expandable sieve is configured to maintain a positioning of the first expandable sieve along a length of the body lumen, while subjected to an irrigation flow in a range of approximately 10 ml/min to 1.0 liter/min, by only a radial force exerted by the first expandable sieve on a wall of the body lumen while the first expandable sieve is in an expanded state.

20. The device of claim 19, wherein the first expandable sieve is axially moveable in at least one direction along the support member.

21. The device of claim 19, wherein the first expandable sieve is configured to substantially prevent a passage of particles with a maximum diameter greater than or equal to 1.0 mm therethrough, and to substantially allow a passage of particles with a maximum diameter less than 1.0 mm therethrough.

22. The device of claim 19, wherein the first expandable sieve is configured to substantially prevent a passage of particles with a maximum diameter greater than or equal to 2.0 mm therethrough, and to substantially allow a passage of particles with a maximum diameter less than 2.0 mm therethrough.

23. The device of claim 19, wherein the sheath is redeployable over the first expandable sieve in the expanded state to return the first expandable sieve substantially back to the collapsed state.

24. The device of claim 19, wherein the first expandable sieve has a compressed diameter of approximately less than 1.0 mm.

25. The device of claim 19, wherein the expanded state of the first expandable sieve is conformable to a body lumen with a diameter in a range of approximately 0.5 mm and 15 mm.

26. The device of claim 19, wherein the first expandable sieve is configured to maintain an opening size less than 2.0 mm throughout an expanded state range of approximately 1.0-13 mm.

27. The device of claim 19, further comprising an anchor located at a distal end of the device.

28. A medical device comprising:
a wire;
a sheath configured to surround at least part of the support member, the sheath being axially moveable in at least one direction along the support member; and
a first expandable sieve configured to be deployed in a body lumen and to be held at least temporarily in a compressed state by the sheath,
wherein the device is configured to accommodate a stent being deployed over a free end of the wire and past the first expandable sieve.

29. A method of removing an obstruction from a body using the device of claim 1, the method comprising:
inserting the wire into a body lumen;
withdrawing the sheath to expand the first expandable sieve from a collapsed state to an expanded state;
inducing a liquid flow in the body lumen; and
straining particulates with a diameter greater than an opening size of the first expandable sieve from the liquid flow in the body lumen using the first expandable sieve, while maintaining a positioning of the first expandable sieve along a length of the body lumen by only a radial force exerted by the first expandable sieve on a wall of the body lumen.

30. A method of removing an obstruction from a body comprising:
inserting an elongated support member and a first expandable sieve into a body lumen, the support member and first expandable sieve at least partially surrounded by a sheath;
positioning the first expandable sieve in a collapsed state distally of the obstruction;
withdrawing the sheath to expand the first expandable sieve from the collapsed state to an expanded state;
straining particulates with a diameter greater than an opening size of the first expandable sieve from a liquid flow in the body lumen using the first expandable sieve; and
advancing the sheath to return the first expandable sieve from the expanded state substantially back to the collapsed state.

31. The method of claim 30, further comprising deploying a stent to the body lumen over the support member and the expandable sieve.

32. The method of claim 30, wherein the obstruction is a kidney stone and the body lumen is a ureter.

33. A method of treating a calcified aggregation in a body comprising:
inserting an elongated support member and a first expandable sieve into a body lumen, the support member and first expandable sieve at least partially surrounded by a sheath;
positioning the first expandable sieve in a collapsed state distally of the calcified aggregation;
withdrawing the sheath to expand the first expandable sieve from the collapsed state to an expanded state;
fragmenting the calcified aggregation; and
straining particulates with a diameter greater than an opening size of the first expandable sieve from a liquid flow in the body lumen using the first expandable sieve while maintaining a positioning of the first expandable sieve along a length of the body lumen by only a radial force exerted by the first expandable sieve on a wall of the body lumen.

34. The method of claim 33, wherein the calcified aggregation is a kidney stone and the body lumen is a ureter.
